(19) United States
(12) Patent Application Publication
(10) Pub. No.: US 2003/0013987 A1
(43) Pub. Date: Jan. 16, 2003

(54) Expandable device for mapping temperature profile of a hollow body organ

(76) Inventor: Vahid Saadat, Saratoga, CA (US)

Correspondence Address: JOHNEY U. HAN, MORRISON & FOERSTER LLP, 755 PAGE MILL ROAD, PALO ALTO,, CA, 94304-1018, US

(21) Appl. No.: 09/904,220

(22) Filed: Jul. 12, 2001

(51) Int. Cl. A61B 5/00

(52) U.S. Cl. 600/549

(57) ABSTRACT

A device for sensing the temperature profile of a hollow body organ includes a catheter and a guidewire. The guidewire is configured as a plurality of helical loops of greater diameter than the catheter when unconstrained. When constrained within the catheter, the guidewire can be advanced to a region of interest in hollow body organ. The catheter can be withdrawn, leaving the guidewire in place in an expanded configuration wherein the helical loops contact the inner wall of the hollow body organ. A thermal sensor is disposed proximate the distal end of the catheter. The distal end of the catheter, being relatively more flexible than the guidewire, traverses a helical path in contact with the inner wall of the hollow body organ, guided by the helical loops of the guidewire.

### TECHNICAL FIELD OF THE INVENTION

This invention relates generally to invasive medical devices and more particularly to devices for sensing the temperature of the interior wall of a hollow body organ such as a blood vessel.

### BACKGROUND OF THE INVENTION

Acute ischemic syndromes involving arterial blood vessels, such as myocardial infarction, or heart attack, and stroke, frequently occur when atherosclerotic plaque ruptures, triggering the formation of blood clots, or thrombosis. Plaque that is inflamed is particularly unstable and vulnerable to disruption, with potentially devastating consequences. Therefore, there is a strong need to detect and locate this type of plaque so that treatment can be initiated before the plaque undergoes disruption and induces subsequent life-threatening clotting.

Various procedures are known for detecting and locating plaque in a blood vessel. Angiography is one such procedure in which X-ray images of blood vessels are generated after a radiopaque dye is injected into the blood stream. This procedure is capable of locating plaque in an artery, but is not capable of revealing whether the plaque is the inflamed, unstable type.

Researchers, acting on the theory that inflammation is a factor in the development of atherosclerosis, have discovered that local variations of temperature along arterial walls can indicate the presence of inflamed plaque. The temperature at the site of inflamation, i.e., the unstable plaque, is elevated relative to adjacent plaque-free arterial walls.

Using a tiny thermal sensor at the end of a catheter, the temperature at multiple locations along an arterial wall were measured in people with and without atherosclerotic arteries. In people free of heart disease, the temperature was substantially homogeneous wherever measured: an average of 0.65 degrees F. above the oral temperature. In people with stable angina, the temperature of their plaques averaged 0.19 degrees F. above the temperature of their unaffected artery walls. The average temperature increase in people with unstable angina was 1.23 degrees F. The increase was 2.65 degrees F. in people who had just suffered a heart attack. Furthermore, temperature variation at different points at the plaque site itself was found to be greatest in people who had just had a heart attack. There was progressively less variation in people with unstable angina and stable angina.

The temperature heterogeneity discussed above can be exploited to detect and locate inflamed, unstable plaque through the use of cavity wall profiling apparatus. Typically, cavity wall profiling apparatus are comprised of temperature indicating probes such as thermocouples, thermistors, fluorescence lifetime measurement systems, resistance thermal devices and infrared measurement devices.

One problem with conventional cavity wall profiling apparatus is that they usually exert an undue amount of force on the region of interest. If the region of interest cannot withstand these forces, it may be damaged. The inside walls of a healthy human artery are vulnerable to such damage. Furthermore, if inflamed, unstable plaque is present it may be ruptured by such forces.

Another problem with conventional cavity wall profiling apparatus is that they can only measure the temperature at one specific location. In order to generate a map of the cavity temperature variation, one would need to move the temperature indicating probe from location to location. This can be very tedious, can increase the risk of damaging the vessel wall or rupturing vulnerable plaque, and may not resolve temporal characteristics of the profile with sufficient resolution. An array of probes could be employed but that could be very big and heavy.

### SUMMARY OF THE INVENTION

According to one aspect of the invention, a device is provided for sensing the temperature profile of a hollow body organ. The device includes a catheter, a guidewire, and a thermal sensor disposed on the catheter proximate the distal end thereof and laterally as well as longitudinally moveable as the distal end travels along the guidewire. The guidewire has an expanded configuration externally of the catheter including a plurality of helical loops of greater diameter than the catheter. The guidewire also has a contracted configuration internally of the catheter and is of a lesser diameter than the catheter. At least the distal end portion of the catheter is more flexible than the guidewire.

According to another aspect of the invention, the device is used by contracting the guidewire elastically and constraining the guidewire within the catheter. The catheter and guidewire are advanced to a region of interest in a hollow body organ. The catheter is withdrawn while securing the guidewire against substantial longitudinal movement relative to the hollow body organ, resulting in the guidewire self-expanding into helical loops in contact with the hollow body organ. As the catheter is withdrawn, the thermal sensor on the catheter traverses a helical path in contact with the hollow body organ, guided by the expanding helical loops of the guidewire. The thermal sensor on the catheter is moved relative to the guidewire to sense the temperature of the hollow body organ at multiple locations.

Further aspects and advantages of the present invention are apparent from the following description of a preferred embodiment referring to the drawings.

### BRIEF DESCRIPTION OF THE DRAWINGS

In the drawings,

FIG. 1 is a perspective, partially cut-away view of an arterial hollow body organ in which a preferred embodiment of the present invention is deployed in one orientation;

FIG. 2 is a perspective, partially cut-away view of an arterial hollow body organ in which the preferred embodiment of FIG. 1 is deployed in another orientation;

FIG. 3 is a perspective, partially cut-away view of an arterial hollow body organ in which the preferred embodiment of FIG. 1 is deployed in yet another orientation;

FIG. 4 is an enlarged perspective view, partially in section, of the preferred embodiment of FIG. 1; and

FIG. 5 is an enlarged view of an alternate embodiment of the present invention showing a rounded sensor element.

### DESCRIPTION OF THE PREFERRED EMBODIMENT

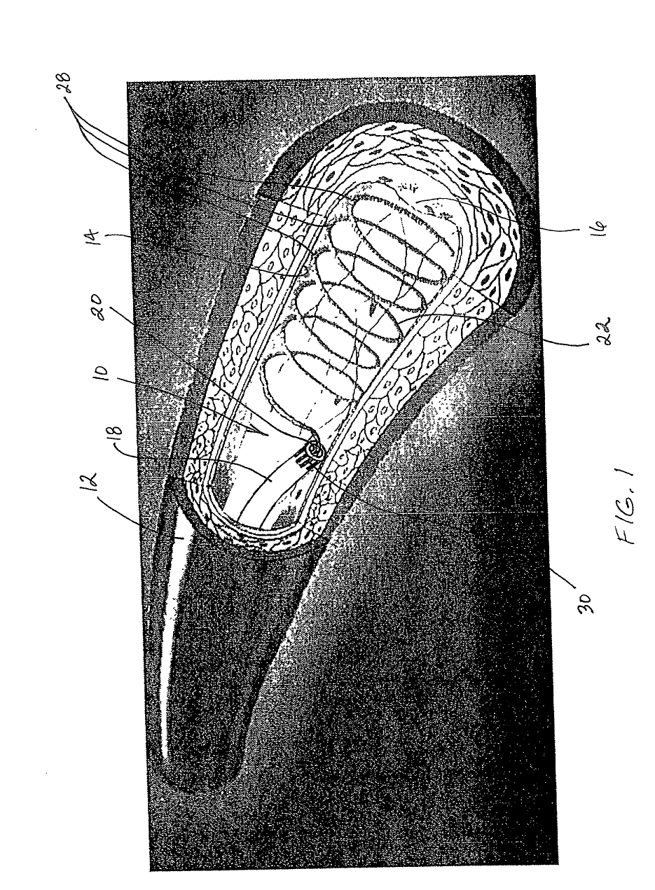

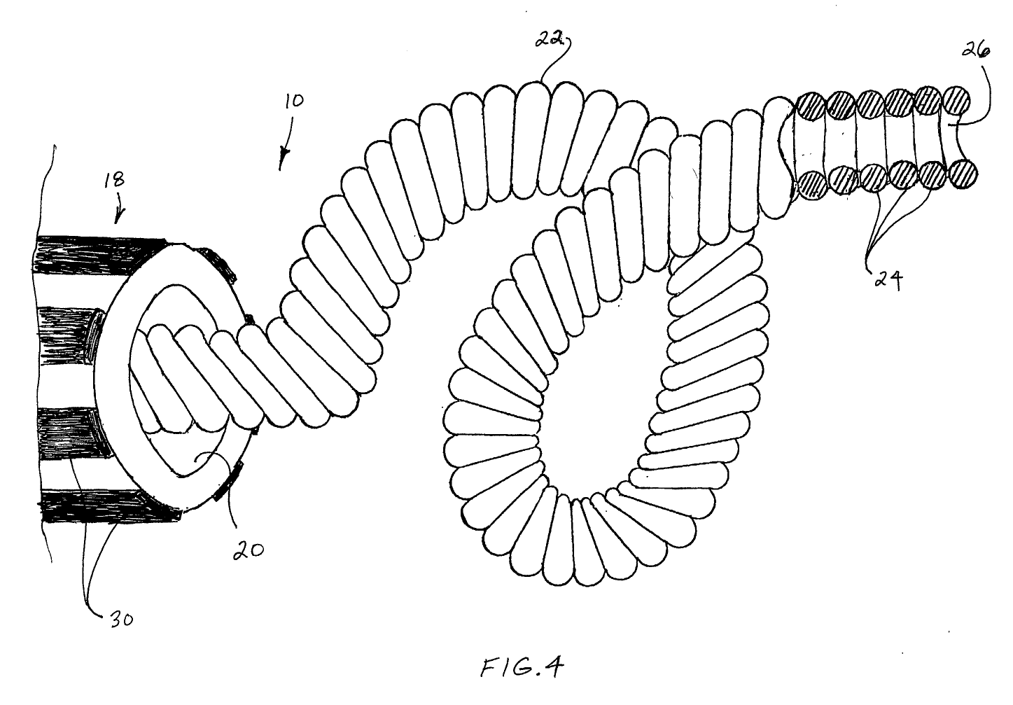

FIGS. 1 through 4 show an expandable device 10 for profiling the wall of a hollow body organ. In FIGS. 1, 2 and 3, device 10 is shown deployed in a hollow body organ comprising an arterial blood vessel 12 having an endothelium 14 forming the inner wall thereof. A plaque 16 is disposed in endothelium 14.

Device 10 includes a lumened catheter 18 having a central lumen 20, a hollow guidewire 22 comprising a tubular helix formed of metal wire 24 or the like in the shape of a coil defining a central lumen 26.

Guidewire 22 is preferably hollow and made of thin wire 24 wound, for example around a mandrel, into small helical coils of desired diameter that lie tightly adjacent one another to form a hollow tube having a central passageway or lumen 26 therethrough. Guidewire 22 has an outer diameter somewhat less than the inner diameter of catheter 18 to permit guidewire 22 to slide freely within the lumen 20 of catheter 18. In addition, guidewire 22, in its relaxed configuration, is shaped as large, loosely spaced helical loops 28. Guidewire 22 can be deformed from this relaxed configuration under force, and when the force is removed guidewire 22 returns to the relaxed, looped configuration.

The self-looping characteristic of guidewire 22 can be accomplished in several ways. One way is to construct guidewire 22 of spring steel that can be deformed into a relatively straight configuration when withdrawn into catheter 18, but which springs back to its looped configuration when extruded from catheter 18 and released from constraint. Another way is to construct guidewire 22 of superelastic nitinol and take advantage of the martensitic transformation properties of nitinol. Guidewire 22 can be inserted into catheter 18 in its straight form and kept cool within the catheter by the injection of cold saline through catheter 18 and over guidewire 22. Upon release of guidewire 22 into the bloodstream, it will warm up and change to its austenite memory shape based on the well-known martensitic transformation by application of heat and putting the material through its transformation temperature.

Guidewire 22 can also be made out of a composite such as a nitinol tube within the guidewire structure. In this fashion, the martensitic or superelastic properties of nitinol can be combined with the spring steel characteristics of the spring and lead to a desirable composition. Other suitable materials for guidewire 22 include copper, constantin, chromel or alumel.

Catheter 18, or at least a distal end portion thereof, is relatively more flexible than guidewire 22, i.e., less stiff, such that the distal end of catheter 18 tends to flex laterally and follow the guidewire 22 laterally as guidewire 22 assumes its looped configuration upon emerging from the constraint of catheter 18. Consequently, as catheter 18 is withdrawn relative to guidewire 18, the distal end of catheter 18 traverses a helical path that follows the just-formed loops 28 as they emerge from catheter 18.

A plurality of thermal sensors 30 are disposed at the distal end of the catheter 18 and situated in spaced relationship to each other around the outside circumference of catheter 18. Conventional conductors or other signal carrying structures (not shown) are provided to convey signals from the thermal sensors along the catheter 18 and out of the proximal end of catheter 18 for connection to appropriate signal processing apparatus that converts the signals to a temperature indication. Thermal sensors 30 can be thermocouples or thermistors, for example.

In use, guidewire 22 is inserted into the lumen 20 of catheter 18 from the proximal end, thereby constraining guidewire 22 into a substantially straight configuration. Using conventional percutaneous insertion techniques, access to the blood vessel 12 is obtained surgically and device 10 is advanced through the blood vessel 12 to the region of interest.

While securing guidewire 22 against movement relative to the patient, and hence the blood vessel 12, catheter 18 is slowly withdrawn such that guidewire 22 emerges from the distal end of catheter 18 and reverts to its looped configuration within the blood vessel 12. Guidewire 22 remains substantially motionless in the axial direction relative to the blood vessel 12 as catheter 18 is withdrawn, with the re-formed loops 28 springing radially outwardly into contact with the vessel wall 14. The relative lack of movement between guidewire 22 and vessel wall 14 alleviates the risk of damage to vessel wall 14 and the risk of rupturing unstable plaque.

As guidewire 22 becomes exposed and loops 28 expand into helical contact with the wall 14 of blood vessel 12, at least one of the thermal sensors 30 circumscribing the distal end of catheter 18 is likewise pushed into contact with the vessel wall 14. Thermal sensors 30 are able to sense the localized temperature of the vessel wall 14 at the region where the thermal sensors 30 are located. By slowly withdrawing catheter 18 relative to guidewire 22, the distal end of catheter 18, by flexing, traverses a helical path around the wall 14 of the blood vessel 12, guided by the relatively stiffer guidewire 22 that is expanding to form loops 28.

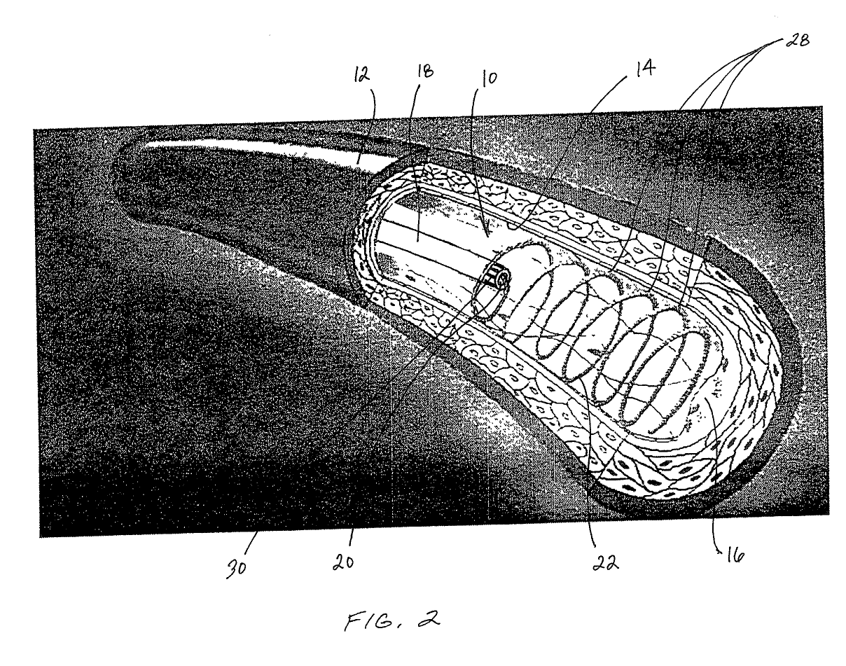

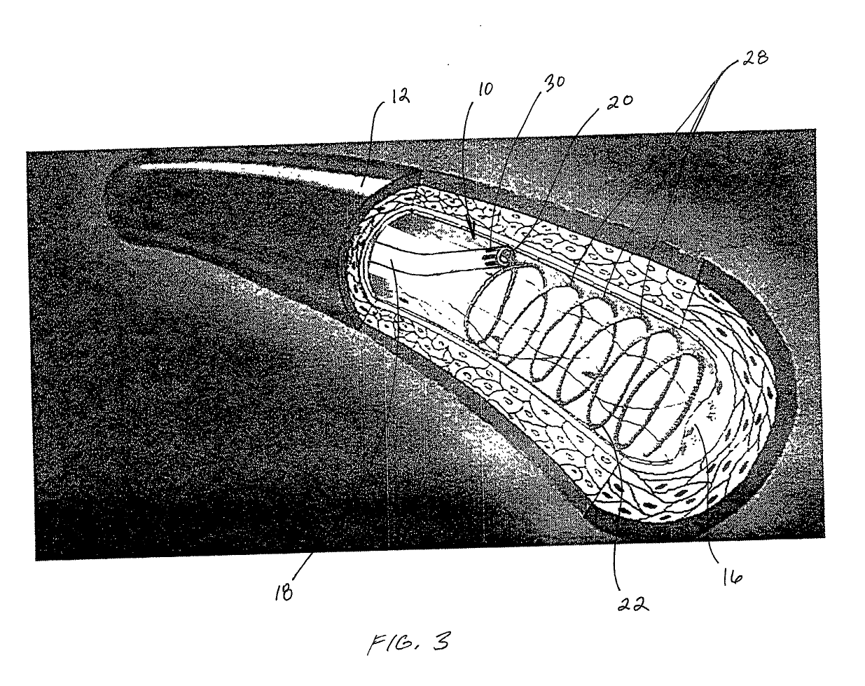

The helical path followed by the distal end of catheter 18 and thermal sensors 30, while being withdrawn relative to guidewire 22, can be envisioned by examining FIGS. 1, 2 and 3. FIG. 1 shows a first orientation of catheter 18 wherein the distal end has been forced, by the expansion of guidewire 22 into loops 28, into contact with a lower portion of the vessel wall 14. FIG. 2 shows a subsequent orientation of catheter 18 after having been withdrawn further relative to guidewire 22. The distal end of catheter 18 has been forced by the expanding loops 28 of guidewire 22 into contact with a rear portion of the vessel wall 14. FIG. 3 shows a further subsequent orientation of catheter 18 after having been withdrawn still further relative to guidewire 22. The distal end of catheter 18 has been forced by the expanding loops 28 of guidewire 22 into contact with an upper portion of the vessel wall 14.

Temperature measurements of different regions of the vessel wall 14 can be taken at intervals as catheter 18 is withdrawn. By withdrawing the catheter 18 at a constant rate, the location of the thermal sensors 30 relative to the distal end of the guidewire 22 can be determined as a function of time, so that a temperature profile of the blood vessel 12 can be mapped.

Once the mapping is completed, the catheter 18 can be pushed forward again while securing guidewire 22 against longitudinal movement. Catheter 18 will thereby re-sheath guidewire 22 and constrain it in a substantially straight configuration for movement to a further region of interest or withdrawal from the blood vessel 12.

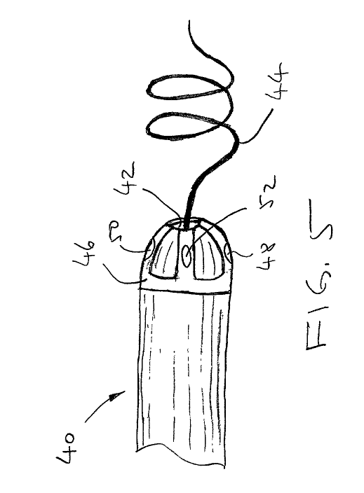

FIG. 5 illustrates an alternate embodiment of the present invention in which a lumened catheter 40 having a guidewire exit aperture 42 is provided at the distal end thereof with a rounded cage or cap 46 that defines the exit aperture 42 and also carries suitable temperature sensing elements or thermal sensors such thermistors 48, 50 and 52, or the like. The rounded configuration of cap 46 minimizes the likelihood of trauma to the surrounding tissue upon contact therewith. A relatively stiff, self-looping guidewire 44 extends outwardly from exit aperture 42 and serves to guide sensing elements 48, 50 and 52 in the same manner as described hereinabove with respect to self-looping guidewire 22.

Although the present invention has been described in detail in terms of preferred embodiments, no limitation on the scope of the invention is intended. The scope of the subject matter in which an exclusive right is claimed is defined in the appended claims.

I claim:

1. A device for sensing the temperature profile of a hollow body organ, comprising:
a catheter having a distal end;
at least one thermal sensor disposed on the catheter proximate the distal end; and
a guidewire being disposable in an expanded configuration externally of the catheter including a plurality of helical loops, and in a contracted configuration internally of the catheter.

2. The device of claim 1, wherein the guidewire comprises a tubular helix.

3. The device of claim 1, wherein the guidewire comprises a material having martensitic transformation properties.

4. The device of claim 3, wherein the guidewire comprises nitinol.

5. The device of claim 1, wherein the guidewire comprises an elastic material.

6. The device of claim 5, wherein the guidewire comprises spring steel.

7. The device of claim 1, wherein the thermal sensor comprises a thermocouple.

8. The device of claim 1, wherein the thermal sensor comprises a thermistor.

9. The device of claim 1, including a plurality of thermal sensors spaced around the catheter at the distal end thereof.

10. The device of claim 1, and further including a conductor for conveying signals from the at least one thermal sensor to the proximal end of the catheter.

11. The device of claim 1, wherein the catheter is relatively more flexible than the guidewire.

12. The device of claim 1 wherein the distal end of the catheter is provided with an apertured rounded cap that carries the thermal sensor.
